(19) United States
(12) Patent Application Publication
(10) Pub. No.: US 2025/0195014 A1
(43) Pub. Date: Jun. 19, 2025

(54) MEDICAL IMAGE PROCESSING APPARATUS, CONTROL METHOD THEREFOR, AND RECORDING MEDIUM FOR MEDICAL IMAGE PROCESSING PROGRAM

(71) Applicant: CANON KABUSHIKI KAISHA, Tokyo (JP)

(72) Inventors: Makiko UENO, Kanagawa (JP); Takeshi NODA, Kanagawa (JP); Akiya NAKAYAMA, Kanagawa (JP)

(21) Appl. No.: 19/072,043

(22) Filed: Mar. 6, 2025

Related U.S. Application Data
(63) Continuation of application No. PCT/JP2023/031810, filed on Aug. 31, 2023.

(30) Foreign Application Priority Data
Sep. 22, 2022 (JP) 2022-151803
Aug. 9, 2023 (JP) 2023-130532

(51) Int. Cl. A61B 6/03; A61B 6/00; A61B 6/42

(52) U.S. Cl. CPC A61B 6/032; A61B 6/4241; A61B 6/5205

(57) ABSTRACT

A medical image processing apparatus includes: an obtaining unit configured to obtain first data obtained by using a first X-ray detection scheme and second data obtained by using a second X-ray detection scheme different from the first X-ray detection scheme; a data generation unit configured to generate third data made similar to the second data by processing the first data or a signal for obtaining the first data; and a registration unit configured to register data based on the first data with data based on the second data based on a result of registration between the data based on the second data and the data based on the third data.

### CROSS-REFERENCE TO RELATED APPLICATIONS

This application is a Continuation of International Patent Application No. PCT/JP2023/031810, filed Aug. 31, 2023, which claims the benefit of Japanese Patent Application No. 2022-151803, filed Sep. 22, 2022 and Japanese Patent Application No. 2023-130532, filed Aug. 9, 2023, all of which are hereby incorporated by reference herein in their entirety.

### BACKGROUND OF THE INVENTION

### Field of the Invention

The present disclosure relates to a medical image processing apparatus, a control method therefor, and a recording medium for a medical image processing program.

### Background Art

In general, CT apparatuses using X-ray detectors based on the integral X-ray detection scheme (integral scheme) are popular. In recent years, CT apparatuses using X-ray detectors based on the photon counting X-ray detection scheme (to be referred to as the photon counting scheme hereinafter) have been put into practical use. PTL 1 discloses a photon counting X-ray CT apparatus based on the count data of X-ray photons collected by using the signal detected by the X-ray detector. The photon counting X-ray CT apparatus can obtain a high-resolution image with a low dose as compared with the integral X-ray CT apparatus based on the integral data obtained by integrating the signals detected by the X-ray detector with a predetermined time width.

### CITATION LIST

### Patent Literature

PTL 1: Japanese Patent Laid-Open No. 2020-127635

It is sometimes desirable that medical images obtained by different X-ray detection schemes be compared with each other when, for example, the CT image obtained by the integral scheme in the past and the CT image newly obtained by the photon counting scheme are preferably compared with each other for follow-up. It is, however, difficult to properly compare the medical images obtained by different X-ray detection schemes, for example, the CT image obtained by the photon counting scheme and the CT image obtained by the integral scheme. This is because differences between two medical images include a mix of differences due to improvements in image quality and differences due to temporal changes of lesions and the like, and the registration between the images obtained by the two schemes is difficult to perform. In addition, the mix of differences makes it difficult to recognize temporal changes.

### SUMMARY OF THE INVENTION

The present disclosure of the present specification provides a technique for further facilitating the comparison between medical images obtained by different X-ray detection schemes.

According to one aspect of the present invention, there is provided a medical image processing apparatus comprising:
an obtaining unit configured to obtain first data obtained by using a first X-ray detection scheme and second data obtained by using a second X-ray detection scheme different from the first X-ray detection scheme;
a data generation unit configured to generate third data made similar to the second data by processing the first data or a signal for obtaining the first data; and
a registration unit configured to register data based on the first data with data based on the second data based on a result of registration between the data based on the second data and the data based on the third data.

According to another aspect of the present invention, there is provided a control method for a medical image processing apparatus, the method comprising:
an obtaining step of obtaining first data obtained by using a first X-ray detection scheme and second data obtained by using a second X-ray detection scheme different from the first X-ray detection scheme;
a data generation step of generating third data made similar to the second data by processing the first data or a signal for obtaining the first data; and
a registration step of registering data based on the first data with data based on the second data based on a result of registration between data based on the second data and data based on the third data.

According to still another aspect of the present invention, there is provided a storage medium storing a program for causing a computer to execute:
an obtaining step of obtaining first data obtained by using a first X-ray detection scheme and second data obtained by using a second X-ray detection scheme different from the first X-ray detection scheme;
a data generation step of generating third data made similar to the second data by processing the first data or a signal for obtaining the first data; and
a registration step of registering data based on the first data with data based on the second data based on a result of registration between data based on the second data and data based on the third data.

Further features of the present invention will become apparent from the following description of exemplary embodiments with reference to the attached drawings.

### BRIEF DESCRIPTION OF THE DRAWINGS

FIG. 1 is a view showing an example of the arrangement of an X-ray CT apparatus according to an embodiment.

FIG. 2 is a view for explaining an X-ray detector according to the embodiment.

FIG. 3A is a flowchart showing medical image processing according to processing example 1.

FIG. 3B is a flowchart showing medical image processing according to processing example 1.

FIG. 3C is a flowchart showing medical image processing according to processing example 2.

FIG. 3D is a flowchart showing medical image processing according to processing example 2.

FIG. 3E is a flowchart showing medical image processing according to processing example 3.

FIG. 3F is a flowchart showing medical image processing according to processing example 4.

FIG. 3G is a flowchart showing learning processing according to processing example 4.

FIG. 3H is a flowchart showing medical image processing according to processing example 5.

FIG. 4A is a view showing a display example of a tomographic image according to processing example 1.

FIG. 4B is a view showing a display example of a tomographic image according to processing example 1.

FIG. 4C is a view showing a display example of information indicating a difference image or different region according to processing example 2.

FIG. 4D is a view showing a display example of information indicating a difference image or different region according to processing example 2.

FIG. 5 is a view showing a display example of an image data set according to the embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

An embodiment of the medical image processing apparatus, the X-ray CT apparatus, and the processing program disclosed in the present specification will be described in detail below. Note that the medical image processing apparatus, the X-ray CT apparatus, and the processing program according to the present application are not limited those described in the following embodiment. The following will exemplify an X-ray detector as a radiation detector and exemplify an X-ray CT apparatus as a radiation diagnosis apparatus.

An X-ray CT apparatus described in the following embodiment is an apparatus that can execute photon counting CT. That is, the X-ray CT apparatus described below is an apparatus that can reconstruct X-ray CT image data with a high S/N ratio by counting the photons generated by X-rays transmitted through a subject by using a photon counting X-ray detector. In addition, the X-ray detector described in the following embodiment is a direct conversion type detector that directly converts X-ray photons into electric charges proportional to the energy.

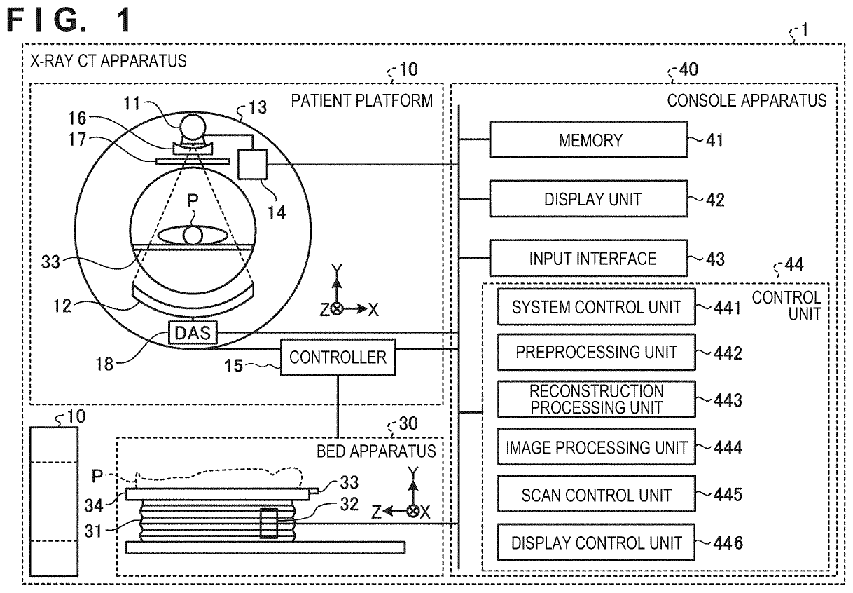

FIG. 1 is a view showing an example of the arrangement of a photon counting X-ray CT apparatus 1. As shown in FIG. 1, the X-ray CT apparatus 1 according to the embodiment includes a patient platform 10, a bed apparatus 30, and a console apparatus 40. Referring to FIG. 1, assume that the direction of the rotational axis of a rotating frame 13 in a non-tilt state or the longitudinal direction of a bed top 33 of the bed apparatus 30 is the Z-axis direction. An axial direction orthogonal to the Z-axis direction and horizontal to the floor surface is the X-axis direction. An axial direction orthogonal to the Z-axis direction and vertical to the floor surface is the Y-axis direction. Although FIG. 1 includes a view rendering the patient platform 10 from a plurality of directions for the sake of explanation, the X-ray CT apparatus 1 according to the embodiment includes the single patient platform 10.

The patient platform 10 includes an X-ray tube 11, an X-ray detector 12, the rotating frame 13, an X-ray high voltage device 14, a controller 15, a wedge 16, a collimator 17, and a DAS 18. Note that DAS is an abbreviation of Data Acquisition System.

The X-ray tube 11 is a vacuum tube having a cathode (filament) that generates thermal electrons and an anode (target) that generates X-rays upon collision with the thermal electrons. The X-ray tube 11 irradiates the anode with thermal electrons from the cathode by applying a high voltage from the X-ray high voltage device 14 to generate X-rays to be applied to a subject P. For example, the X-ray tube 11 includes a rotating anode type X-ray tube that generates X-rays by irradiating the rotating anode with thermal electrons.

The rotating frame 13 is an annular frame that supports the X-ray tube 11 and the X-ray detector 12 so as to make them face each other and rotates the X-ray tube 11 and the X-ray detector 12 under the control of the controller 15. For example, the rotating frame 13 is a casting made of aluminum. Note that the rotating frame 13 can also support the X-ray high voltage device 14, the wedge 16, the collimator 17, the DAS 18, and the like in addition to the X-ray tube 11 and the X-ray detector 12. In addition, the rotating frame 13 can support various components (not shown in FIG. 1).

The wedge 16 is a filter for adjusting the dose of X-rays emitted from the X-ray tube 11. More specifically, the wedge 16 is a filter that transmits and attenuates the X-rays emitted from the 11 so as to make the X-rays emitted from the X-ray tube 11 to the subject P have a predetermined distribution. For example, the wedge 16 is a filter obtained by processing aluminum or the like to have a predetermined target angle or a predetermined thickness and includes a wedge filter or a bow-tie filter.

The collimator 17 narrows the irradiation range of X-rays transmitted through the wedge 16. The collimator 17 has a slit formed by combining a plurality of lead plates and the like. Note that the collimator 17 is sometimes called an X-ray aperture. FIG. 1 shows a case where the wedge 16 is placed between the X-ray tube 11 and the collimator 17. However, the collimator 17 may be placed between the X-ray tube 11 and the wedge 16. In this case, the wedge 16 is irradiated with X-rays from the X-ray tube 11 and is configured to transmit and attenuate X-rays whose irradiation range is limited by the collimator 17.

The X-ray high voltage device 14 includes a high voltage generation unit that generates a high voltage to be applied to the X-ray tube 11 and an X-ray control unit that controls an output voltage corresponding to the X-rays generated by the X-ray tube 11. A high voltage generation scheme used by the high voltage generation unit may be, for example, a transformer scheme or an inverter scheme. Note that the X-ray high voltage device 14 may be provided on the rotating frame 13 or a fixed frame (not shown).

The controller 15 includes a processing circuit having a central processing unit (CPU) and the like and a drive mechanism such as a motor and an actuator. The controller 15 receives a signal from an input interface 43 of the console apparatus 40 and controls the operations of the patient platform 10 and the bed apparatus 30. For example, the controller 15 controls the rotation of the rotating frame 13, the tilt of the patient platform 10, the operation of the bed top 33 by the bed apparatus 30, and the like. For example, to tilt the patient platform 10, the controller 15 rotates the rotating frame 13 about an axis parallel to the X-axis direction in accordance with the inclination angle (tilt angle) information input from the input interface 43. Note that the controller 15 may be provided in the patient platform 10 or the console apparatus 40.

The X-ray detector 12 is a photon counting detector that outputs a signal that allows the measurement of the energy value of X-ray photons every time the X-ray photons enter. The X-ray photons detected by the X-ray detector 12 are, for example, the X-ray photons emitted from the X-ray tube 11 and transmitted through the subject P. The X-ray detector 12 has a plurality of detection elements that output a one-pulse electrical signal (analog signal) every time X-ray photons enter. For example, the X-ray detector 12 has a structure in which a plurality of X-ray detection element rows, each having a plurality of X-ray detection elements (to be also simply referred to as “detection elements” hereinafter) arrayed in the channel direction along one arc centered on the focus of the X-ray tube 11, are arrayed in the slice direction. The detection elements of the X-ray detector 12 can count the X-ray photons entering the detection elements by counting electrical signals (pulses). The detection elements can also measure the energy value of the X-ray photons that have caused the signals to be output by performing arithmetic processing with respect to this signal.

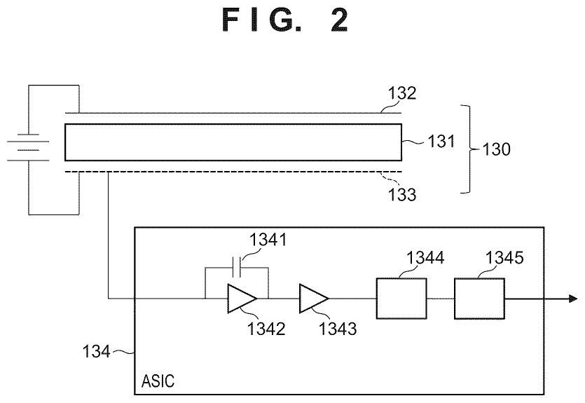

FIG. 2 is a schematic view showing the arrangement of part of the X-ray detector 12 according to this embodiment. The X-ray detector 12 is a photon counting detector and includes, as shown in FIG. 2, a detection element 130 and an ASIC 134 connected to the detection element 130 and configured to count the X-ray photons detected by the detection element 130. Note that ASIC is an abbreviation of Application Specific Integration Circuit. The X-ray detector 12 according to the embodiment is a direct conversion type detector that directly converts incident X-ray photons into an electrical signal. FIG. 2 shows one of the plurality of detection elements constituting the X-ray detector 12.

The detection element 130 includes a semiconductor 131, a cathode electrode 132, and a plurality of anode electrodes 133. In this case, the semiconductor 131 is a semiconductor such as cadmium telluride (CdTe) or cadmium zinc telluride (CdZnTe). Each of the plurality of anode electrodes 133 corresponds to a detection pixel (to be also referred to as a “pixel”). When X-ray photons enter the detection element 130, the detection element 130 directly converts the X-rays entering the detection element 130 into electric charges and outputs them from the anode electrode 133 corresponding to the incident position of X-rays to the ASIC 134.

The ASIC 134 is provided for each of the plurality of anode electrodes 133 (that is, each detection pixel). The ASIC 134 counts incident X-ray photons for each detection pixel of the detection element 130 by discriminating the electric charges output from the detection element 130. The ASIC 134 also measures the energy of the counted X-ray photons by performing arithmetic processing based on the magnitude of each electric charge. The ASIC 134 outputs the count result of the X-ray photons and/or the energy measurement result of the X-ray photons as digital data to the DAS 18.

The ASIC 134 includes, for example, a capacitor 1341, an amplification circuit 1342, a wave shaping circuit 1343, a comparator circuit 1344, and a counter 1345. The capacitor 1341 accumulates the electric charges output from the detection element 130. The amplification circuit 1342 integrates the electric charges collected in the capacitor 1341 in response to the X-ray photons entering the detection element 130, amplifies the electric charges, and outputs the result as a pulse signal of an electric quantity. The wave height or area of this pulse signal has a correlation with the energy of the photons.

The amplification circuit 1342 includes, for example, an amplifier. The amplifier may be, for example, a single end amplifier or a differential amplifier. In the case of the single end amplifier, the amplifier is grounded and configured to amplify the potential difference between the ground potential (ground) and the potential indicated by the electrical signal output from the detection element 130. In the case of the differential amplifier, the positive input (+) of the amplifier is connected to the detection element 130, and the negative input (−) is grounded. The differential amplifier amplifies the potential difference between the potential indicated by an electrical signal from the detection element 130, which is input to the positive input, and the ground potential indicated by a signal input to the negative input.

The wave shaping circuit 1343 adjusts the frequency characteristics of the pulse signal output from the amplification circuit 1342 and applies a gain and an offset to the signal, thereby shaping the waveform of the pulse signal. The comparator circuit 1344 compares the wave height or area of a response pulse signal with respect to incident photons with a threshold set in advance in accordance with a plurality of energy bands to be discriminated and outputs the comparison result with respect to the threshold to the counter 1345 on the subsequent stage. The counter 1345 counts the discrimination result of the waveforms of response pulses for each corresponding energy band and outputs the count result of photons as digital data to the DAS 18. For example, the counter 1345 generates digital data indicating the count result of X-ray photons in each of a plurality of energy bands and outputs the generated digital data to the DAS 18.

With the above arrangement, the X-ray detector 12 detects X-ray photons and obtains energy information. Obviously, the X-ray detector 12 described above may be provided with a grid on the X-ray incident side of the detection element 130. The X-ray detector 12 may be an indirect conversion type photon counting detector including a grid, a scintillator array, and an optical sensor array. The scintillator array includes a plurality of scintillators. Each scintillator is formed of a scintillator crystal that outputs the number of photons corresponding to incident X-ray energy. The grid is formed of an X-ray shielding plate arranged on the X-ray incident surface of the scintillator array and having a function of absorbing scattered X-rays. The optical sensor array has a function of converting the light into an electrical signal corresponding to the amount of light from the scintillator and is formed of, for example, an optical sensor such as a photoelectron multiplier. In this case, the optical sensor is, for example, a photodiode (PD), an avalanche photodiode (APD), or silicon photomultipliers (SiPM).

The DAS 18 generates detection data based on the count result input from the X-ray detector 12. The detection data is, for example, a sinogram. The sinogram is data having an array of the results obtained by performing count processing of the X-ray photons entering the detection element 130 at each position of the X-ray tube 11. The sinogram is data having count processing results arrayed in a two-dimensional orthogonal coordinate system with the view direction and the channel direction serving as axes. The DAS 18 generates, for example, a sinogram for each column in the slice direction in the X-ray detector 12. In this case, a count processing result is data assigning an X-ray photon count for each energy bin. For example, the DAS 18 counts photons (X-ray photons) originating from the X-rays emitted from the X-ray tube 11 and transmitted through the subject P and discriminates the energies of the counted X-ray photons to obtain count processing results. The DAS 18 transfers the generated detection data to the console apparatus 40. The DAS 18 is implemented by, for example, a processor.

The data generated by the DAS 18 is transmitted from a transmitter having a light-emitting diode and provided on the rotating frame 13 to a receiver having a photodiode (PD) by optical communication and is transferred to the console apparatus 40. The receiver is provided on, for example, a non-rotation portion of the patient platform 10, such as a fixed frame rotatably supporting the rotating frame 13. Note that FIG. 1 omits the illustration of the transmitter and the receiver. In addition, a method of transmitting data from the rotating frame 13 to the non-rotation portion of the patient platform 10 is not limited to optical communication described above. The data generated by the DAS 18 may be transmitted by using any non-contact type data transmission scheme or a contact type data transmission scheme.

The bed apparatus 30 is an apparatus that moves the subject P to be imaged while the subject P is placed on the apparatus and includes a base 31, a bed drive device 32, the bed top 33, and a support frame 34. The base 31 is a housing that supports the support frame 34 so as to allow it to move in the vertical direction. The bed drive device 32 is a drive mechanism that moves the bed top 33 on which the subject P is placed in the long-axis direction of the bed top 33 and includes a motor and an actuator. The bed top 33 provided on the upper surface of the support frame 34 is a plate on which the subject P is placed. Note that the bed drive device 32 may move the support frame 34, in addition to the bed top 33, in the long-axis direction of the bed top 33.

The console apparatus 40 includes a memory 41, a display unit 42, the input interface 43, and a control unit 44. Although this embodiment will exemplify the console apparatus 40 and the patient platform 10 as separate arrangements, the patient platform 10 may include the console apparatus 40 or some of the constituent elements of the console apparatus 40.

The memory 41 is implemented by, for example, a random access memory (RAM), a semiconductor memory element such as a flash memory, a hard disk, or an optical disk. The memory 41 stores, for example, projection data and CT image data. In addition, for example, the memory 41 stores a program for making the control unit included in the X-ray CT apparatus 1 implement its function. Note that the memory 41 may be implemented by a server group (cloud) connected to the X-ray CT apparatus 1 via a network.

The display unit 42 displays various types of information. For example, the display unit 42 displays various types of images generated by the control unit 44 and displays a graphical user interface (GUI) for receiving various types of operations from the operator. For example, the display unit 42 is a liquid crystal display or a cathode ray tube (CRT). The display unit 42 may be of a desk top type or a tablet terminal that can perform wireless communication with the console apparatus 40.

The input interface 43 functions as an instruction acceptance unit that accepts various types of input operations from the operator, converts the accepted input operations into electrical signals, and outputs the signals to the control unit 44. In addition, for example, the input interface 43 accepts, from the operator, input operations for reconstruction conditions in reconstructing CT image data and image processing conditions in generating postprocessed images from CT image data. For example, the input interface 43 can be implemented by a mouse, a keyboard, a trackball, switches, buttons, a joystick, a touch pad that performs an input operation upon contact with an operation surface, a touch screen obtained by integrating a display screen and a touch pad, a non-contact input unit using an optical sensor, a speech input unit, and the like. Note that if the input interface 43 is a touch screen, the input interface 43 can also have the function of the display unit 42. The input interface 43 may be provided on the patient platform 10. The input interface 43 may be formed of a tablet terminal that can perform wireless communication with the console apparatus 40. The input interface 43 is not limited to the one including physical operation components such as a mouse and a keyboard. For example, the input interface 43 may be configured to accept an electrical signal corresponding to an input operation from an external input device provided separately from the console apparatus 40 and perform processing to output the electrical signal to the control unit 44.

The control unit 44 controls the overall operation of the X-ray CT apparatus 1. The control unit 44 executes, for example, the functions of a system control unit 441, a preprocessing unit 442, a reconstruction processing unit 443, an image processing unit 444, a scan control unit 445, and a display control unit 446. The functions executed by the respective functional units of the control unit 44 shown in FIG. 1 are recorded in the memory 41 in the form of, for example, programs that can be executed by the computer. The control unit 44 is, for example, a processor that reads out and executes each program from the memory 41 to implement the function corresponding to each readout program. In other words, the control unit 44 in a state in which each program is read out will implement each functional unit indicated in the control unit 44 in FIG. 1. Although FIG. 1 shows the case where the single control unit 44 implements each of the functions of the system control unit 441, the preprocessing unit 442, the reconstruction processing unit 443, the image processing unit 444, the scan control unit 445, and the display control unit 446, the present disclosure is not limited to this. For example, the control unit 44 may be formed by combining a plurality of independent processors, and each processor may implement each function by executing each program. In addition, the respective functions of the control unit 44 may be implemented by properly being distributed or integrated into a single functional unit or a plurality of functional units.

The system control unit 441 controls each type of function of the control unit 44 based on an input operation accepted from the operator via the input interface 43. The preprocessing unit 442 generates projection data by performing preprocessing such as logarithmic conversion processing, offset correction processing, sensitivity correction processing between channels, and beam hardening correction for the detection data output from the DAS 18. The reconstruction processing unit 443 generates CT image data (volume data) by performing reconstruction processing using a filter correction back projection method, a sequential approximate reconstruction method, or the like with respect to the projection data generated by the preprocessing unit 442. The reconstruction processing unit 443 stores the reconstructed CT image data in the memory 41.

The projection data generated from the count result obtained by the photon counting X-ray CT apparatus 1 includes the information of the energy of the X-rays attenuated by being transmitted through the subject P. This enables the reconstruction processing unit 443 to reconstruct, for example, the CT image data of a specific energy component. In addition, the reconstruction processing unit 443 can reconstruct, for example, the CT image data of each of a plurality of energy components.

The reconstruction processing unit 443 can generate, for example, image data obtained by superimposing a plurality of CT image data color-coded in accordance with energy components by assigning color tones corresponding to the energy components to the respective pixels of the CT image data of the respective energy components. In addition, the reconstruction processing unit 443 can generate image data that allows the identification of a substance by using the K absorption end unique to the substance. Other image data that can be generated by the reconstruction processing unit 443 includes monochromatic X-ray image data, density image data, and effective atomic number image data.

In order to reconstruct CT image data, projection data corresponding to one rotation around a subject, i.e., 360°, is required, or (180°+fan angle) projection data is required in the half scan method. Either of these reconstruction schemes can be applied to this embodiment. For the sake of simple explanation, assume that the reconstruction (full scan reconstruction) scheme is used, which reconstructs CT image data by using projection data corresponding to 360° which is obtained by making the X-ray tube 11 and the X-ray detector 12 make one rotation around the subject.

The image processing unit 444 converts the CT image data generated by the reconstruction processing unit 443 into image data such as a tomographic image of an arbitrary section or a three-dimensional image based on an input operation from the operator which is accepted via the input interface 43. The image processing unit 444 stores the converted image data in the memory 41. The image processing unit 444 performs medical image processing to be described later in processing examples 1 to 5.

The scan control unit 445 controls CT scan performed by the patient platform 10. For example, the scan control unit 445 controls count result collection processing in the patient platform 10 by controlling the operations of the X-ray high voltage device 14, the X-ray detector 12, the controller 15, the DAS 18, and the bed drive device 32. For example, the scan control unit 445 controls projection data collection processing in imaging for collecting a positioning image (scan image) and real imaging (scan) for collecting an image used for diagnosis. The display control unit 446 performs control to display various types of image data stored in the memory 41 on the display unit 42.

The following is a description of processing for displaying the tomographic image based on the tomographic image based on the projection data obtained by the photon counting X-ray detection scheme and the projection data obtained by the integral X-ray detection scheme. Note that the integral X-ray detection scheme is an X-ray detection scheme of generating a CT image based on the integral data obtained by integrating the signals detected by the X-ray detector with a predetermined time width. Note that the data obtained by the photon counting X-ray detection scheme (the “data” described here includes images such as tomographic images) is sometimes called the data “obtained by the photon counting scheme” or “PC type” data. Likewise, the data obtained by the integral X-ray detection scheme is sometimes called the data “obtained by the integral scheme” or “integral type” data. According to each processing example described below, it is possible to obtain display suitable for medical diagnosis by reducing the positional shift and the image quality difference between the tomographic image obtained by the photon counting scheme and the tomographic image obtained by the integral scheme.

### Processing Example 1

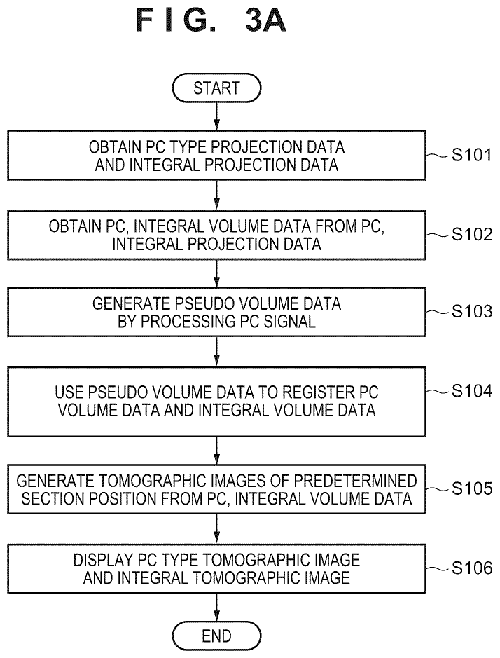

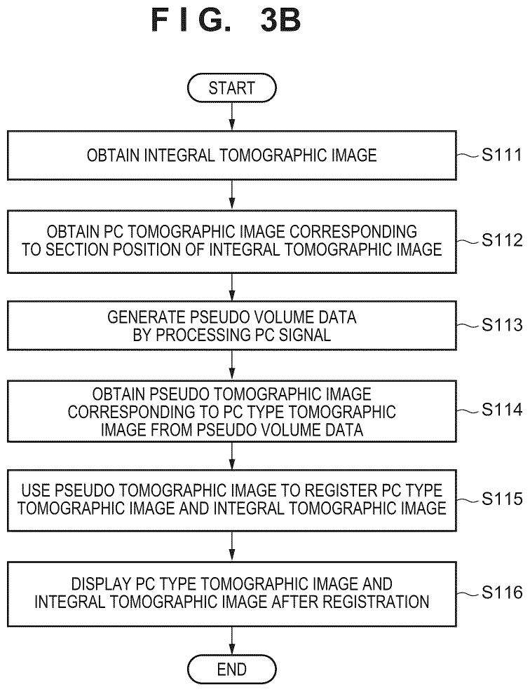

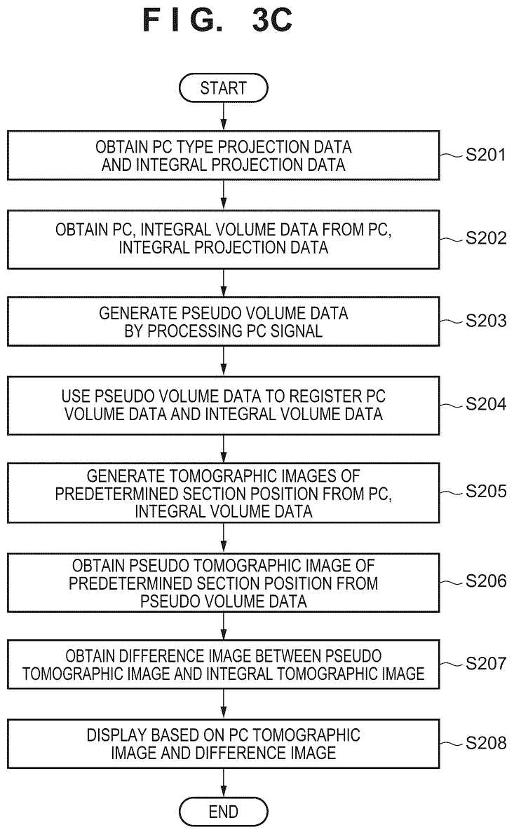

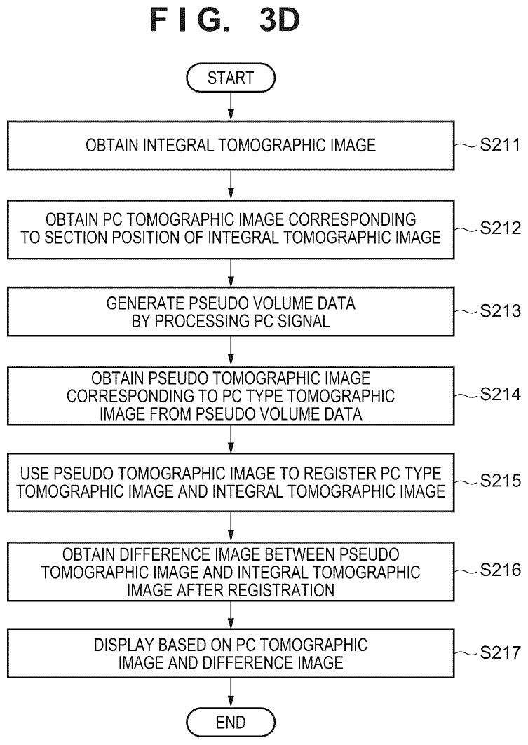

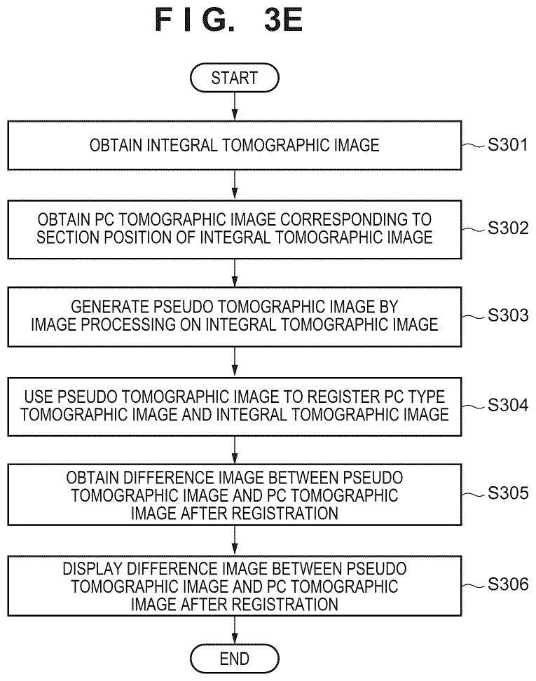

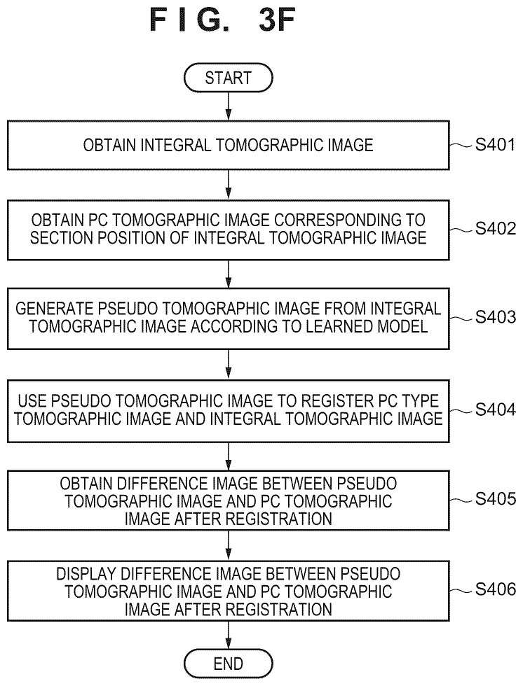

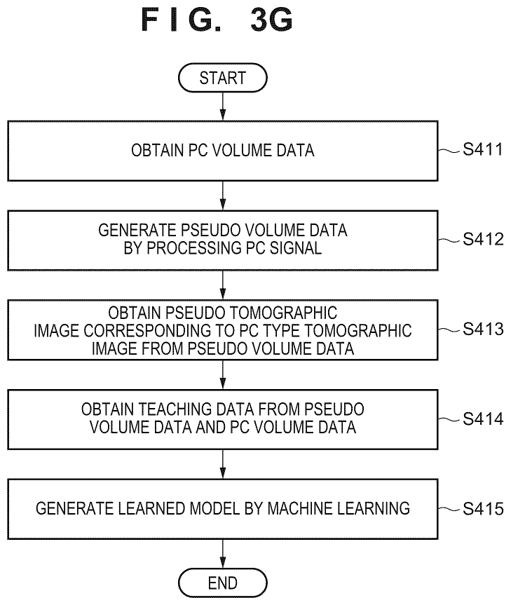

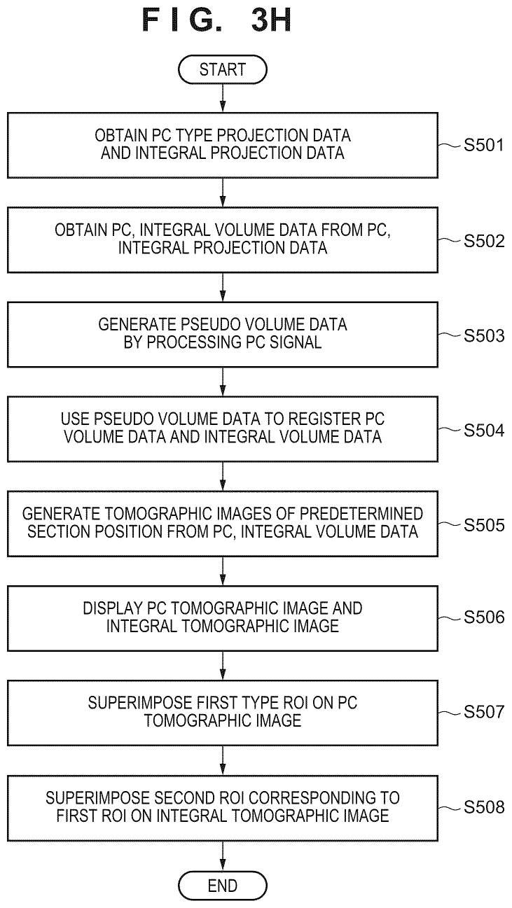

In processing example 1, third data is generated by processing a signal for obtaining first data so as to reduce the difference in image quality between the image obtained based on the first data obtained by the photon counting scheme by the X-ray CT apparatus 1 and the image obtained based on the second data obtained by the integral scheme in a past examination. The generated third data is used for registration or radiogram interpretation. FIG. 3A is a flowchart for explaining the processing performed by the image processing unit 444 and the display control unit 446 in processing example 1.

In step S101, the image processing unit 444 obtains the first projection data that is the first data obtained by using the first X-ray detection scheme and the second projection data that is the second data obtained by using the second X-ray detection scheme as an X-ray detection scheme different from the first X-ray detection scheme. In this case, assuming that the first projection data and the second projection data are obtained in examinations on different dates and times, the first projection data is the data (to be referred to as the PC type projection data hereinafter) obtained by the photon counting CT apparatus (to be referred to as a PC type CT apparatus hereinafter) in the current examination, and the second projection data is the data (to be referred as the integral projection data hereinafter) obtained by the CT apparatus based on the integral scheme (to be referred to as the integral CT apparatus hereinafter) in a past examination. Note that integral projection data may be the data stored in a memory (the memory 41 or another memory) in the console apparatus 40 or the data stored in an external device such as a server connected to the console apparatus 40.

In step S102, the image processing unit 444 generates volume data by performing back projection processing and reconstruction processing for each of PC type projection data and integral projection data by using the reconstruction processing unit 443. In this case, the image processing unit 444 obtains the volume data (to be referred to as the PC type volume data hereinafter) from the PC type projection data that is the data based on the first data and the volume data (to be referred to as integral volume data hereinafter) generated from integral projection data that is the data based on the second data by performing reconstruction processing using the filter correction back projection method for each of PC type projection data and integral projection data.

In step S103, in the current examination, the image processing unit 444 generates the third projection data (to be referred to as the pseudo projection data hereinafter) that is the third data made similar to the projection data obtained by the integral scheme from the signal detected by the X-ray detector 12. In this case, the image processing unit 444 functions as a data generation unit. The image processing unit 444 generates the third volume data (to be referred to as the pseudo volume data hereinafter) that is the data based on the third data by performing back projection processing and reconstruction processing for pseudo projection data by using the reconstruction processing unit 443. The signal detected by the X-ray detector 12 is a signal used to generate photon counting projection data (PC type projection data) that is the detection data output from the DAS 18. For example, pseudo volume data can be obtained in the following manner. First of all, the DAS 18 outputs detection data including independent information including a photon count and an energy value. The preprocessing unit 442 obtains the photon count and the energy value from the detection data and calculates the integral value of the product of these values. The image processing unit 444 generates pseudo projection data by using the integral value and generates pseudo volume data by performing reconstruction processing based on the filter correction back projection method by using the reconstruction processing unit 443. In this manner, pseudo volume data made similar to the volume data obtained by the integral X-ray CT apparatus is generated. According to the above description, the preprocessing unit 442 calculates an integral value. However, the DAS 18 may calculate an integral value and output it as part of detection data.

In step S104, the image processing unit 444 registers the integral volume data obtained in step S102 with the pseudo volume data obtained in step S103. The image processing unit 444 registers the PC type volume data and the integral volume data based on the registration result (for example, the positional shift amount between the integral volume data and the pseudo volume data). More specifically, for example, two volume data are registered in the following manner. The image processing unit 444 extracts feature points (preferably, a plurality of feature points) from one volume data of two volume data to be registered (PC type volume data and integral volume data) and searches for corresponding feature points in the other volume data. Feature points may include SIFT, HOG, and SURF. In addition, the image processing unit 444 may extract characteristic portions of an image as feature points by performing edge detection processing or the like. In order to search for corresponding feature points, template matching, that is, sum of square difference (SSD), normalized correlation, and the like can be used. The image processing unit 444 then registers the two volume data by performing affine conversion with respect to one of the volume data so as to minimize the position differences with respect to the corresponding feature points by using the least square method or the like. In this case, if the positions of the feature points greatly differ from each other, triangular meshes having the feature points as lattice points may be formed, and registration may be performed by using a nonlinear conversion such as local affine conversion of performing affine conversion with respect to each triangular mesh. Nonlinear conversion includes, for example, free-form deformation (FFD) using B-spline. This conversion amount is used as the above positional shift amount.

In step S105, the image processing unit 444 generates tomographic images at the same section position from the PC type volume data and the integral volume data after registration. The tomographic image obtained from PC type volume data will be referred to as a PC type tomographic image, and the tomographic image obtained from integral volume data will be referred to as an integral tomographic image hereinafter. Note that a section position for the generation of tomographic images can be designated by the operator via the input interface 43. In step S106, the display control unit 446 displays the two tomographic images of the sections at the same position, which are generated in step S105, on the display unit 42. Note that the operator described here may be an examiner.

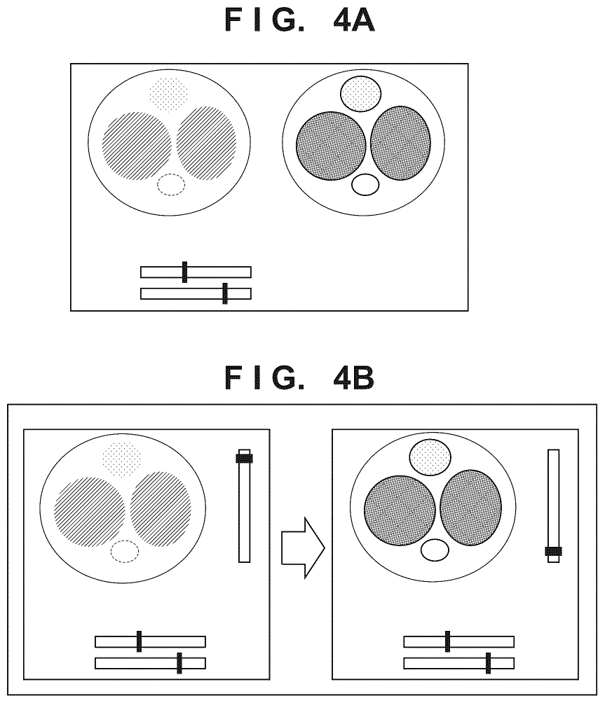

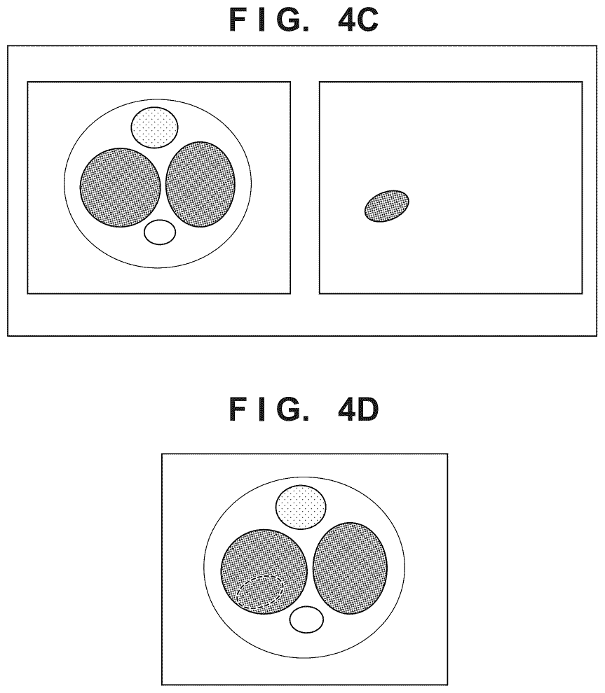

FIG. 4A is a schematic view displaying the tomographic images of sections at the same position based on PC type volume data and integral volume data. For example, the right side in FIG. 4A(a) is the PC type tomographic image of a predetermined section in the PC type volume data, and the left side is the integral tomographic image at the same position as that of the predetermined section in the integral volume data. A display method for these two tomographic images may include a method of performing pair display (parallel display) of simultaneously displaying the tomographic images at left and right positions or upper and lower positions, as shown in FIG. 4A(a). In this case, the two tomographic images may be displayed on the same screen or different screens as long as they are displayed at the same time. In addition, the display of each image may be started or ended at different timings as long as they are displayed at the same time. Alternatively, as shown in FIG. 4A(b), stack display may be performed such that the two tomographic images are individually displayed by page switching. Since the two tomographic images displayed in FIGS. 4A(a) and 4A(b) are properly registered, the operator can easily check a temporal change in a target region such as a lesion. In addition, at this time, the tomographic images (to be referred to as pseudo tomographic images hereinafter) of sections at the above positions in the pseudo volume data (the same positions in the PC type tomographic image and the integral tomographic image) may be displayed. For example, in parallel display like that shown in FIG. 4A(a), the PC type tomographic image may be replaced with a pseudo tomographic image, and the pseudo tomographic image and an integral tomographic image may be displayed in parallel. In stack display (switching display) like that shown in FIG. 4A(b), the PC type tomographic image may be replaced with a pseudo tomographic image, and switching display of the pseudo tomographic image and the integral tomographic image may be performed. This allows the operator to compare a past tomographic image with a current tomographic image with similar image quality.

In this case, the image processing unit 444 obtains PC type projection data and integral projection data in step S101 and generates PC type volume data and integral volume data in step S102. However, [symbol] :22 limitation is not made thereto. If “the data based on the a-th data” (a is a natural number) is written, “the data based on the a-th data” may be the a-th data or the data generated from the a-th data. Accordingly, for example, if integral volume data is externally obtained as the second data, only PC type projection data as the first data is obtained in step S101. In step S102, PC type volume data is generated as the data based on the first data, and the integral volume data obtained above is used as the data based on the second data. If the image processing unit 444 externally obtains PC type volume data as the first data and integral volume data as the second data, the image processing unit 444 skips obtaining projection data and generating volume data. The data based on the first data becomes obtained PC type volume data, and the data based on the second data becomes obtained integral volume data. In either case, as in the above case, a signal for obtaining the first data (PC type projection data or PC type volume data) is processed in step S103 to generate pseudo projection data that is the third data made similar to the projection data obtained by the integral scheme. Back projection processing and reconstruction processing are then performed for the generated pseudo projection data to generate pseudo volume data that is the data based on the third data.

The only data obtained by the integral CT apparatus in a past examination is sometimes an integral tomographic image. In this case, no integral projection data or integral volume data can be obtained. Accordingly, in place of the registration of volume data, it is possible to obtain a corresponding PC type tomographic image from the information of the tomographic position of an integral tomographic image and perform registration between the tomographic images. Therefore, a combination of the data based on the first data and the data based on the second data forms a PC type tomographic image and an integral tomographic image. The processing in this case will be described with reference to the flowchart of FIG. 3B.

In step S111, the image processing unit 444 obtains an integral tomographic image as the second data. Note that integral tomographic images may be stored in a memory (the memory 41 or another memory) in the console apparatus 40 or stored in an external device such as a server connected to the console apparatus 40. In step S112, the image processing unit 444 obtains a PC type tomographic image that is the first data corresponding to the section position of the integral tomographic image based on the information of the section position of the integral tomographic image obtained in step S111. As described above, the PC type tomographic image is obtained from the PC type volume data generated from the PC type projection data obtained from the X-ray CT apparatus 1 in the current examination. In step S113, the image processing unit 444 generates pseudo volume data by processing the signal based on the photon counting scheme. This processing is similar to step S103. In step S114, the image processing unit 444 obtains a tomographic image corresponding to the section position of the PC type tomographic image obtained in step S112 as a pseudo tomographic image that is the third data. In step S115, the image processing unit 444 registers the integral tomographic image obtained in step S111 by using the pseudo tomographic image obtained in step S114 with the PC type tomographic image obtained in step S112. For the registration between tomographic images, it is possible to use, for example, a method of extracting feature points as in the volume data registration or a method using a value representing partial or total correlation between tomographic images. The image processing unit 444 then registers the PC type tomographic image obtained in step S112 with the integral tomographic image obtained in step S111 based on the registration result. In step S116, the display control unit 446 displays the PC type tomographic image and the integral tomographic image registered with each other in step S115. As in step S106, the display control unit 446 may perform display using a pseudo tomographic image, for example, displaying a pseudo tomographic image in place of a PC type tomographic image.

### Processing Example 2

Processing example 1 has described the example of obtaining and displaying a PC tomographic image and an integral tomographic image at a predetermined section position from the PC type volume data and the integral volume data registered with each other by using the pseudo volume data generated from pseudo projection data. Processing example 2 describes an example of generating a difference image between these tomographic images and displaying the difference image or a difference region. FIG. 3C is a flowchart for explaining processing example 2.

Steps S201 to S205 are similar to steps S101 to S105 in processing example 1 (FIG. 3A). As described above, a PC type tomographic image is a tomographic image obtained from the PC type volume data generated based on the PC type projection data obtained by the photon counting scheme in the current examination. In addition, an integral tomographic image is a tomographic image obtained from the integral volume data generated based on the integral projection data obtained by the integral scheme in a past examination.

In step S206, the image processing unit 444 generates, from the pseudo volume data generated in step S203, a PC type tomographic image and a tomographic image (pseudo tomographic image) at the same section position where an integral tomographic image is generated. In step S207, the image processing unit 444 generates an image representing the difference between the integral CT tomographic image and the pseudo tomographic image. In this embodiment, the image processing unit 444 generates a difference image representing a region (difference region) in an identifiable manner, as an example of an image representing a difference, which represents the difference between the integral CT tomographic image and the pseudo tomographic image. The difference image may be an image obtained by calculating the differences in pixel value between the integral CT tomographic image and the pseudo tomographic image, as indicated by, for example, the image on the right side in FIG. 4B(a), or an image having information indicating a difference region reflected in a PC type tomographic image. Note that another example of an image representing a difference may be an image representing the ratios between the respective pixels of two tomographic images. In step S208, the display control unit 446 displays the difference image generated in step S207 on the display unit 42. In this case, for example, the PC type tomographic image (the image on the left side in FIG. 4B(a)) and the difference image (the image on the right side in FIG. 4B(a)) may be displayed in parallel as shown in FIG. 4B(a) or a difference image superimposed on an information PC type tomographic image indicating a difference region may be displayed as shown in FIG. 4B(b). In step S207, if the luminance difference between the first tomographic image and the second tomographic image is large, the image processing unit 444 may generate a difference image or calculate a difference region upon adjusting the luminance value of the pseudo image. Obviously, the display described in step S106 (the display of the PC type tomographic image, the integral tomographic image, and the pseudo tomographic image) may be used in combination.

As described above, in processing example 2, it is possible to generate an image that indicates a temporal change and reduces the influence of the difference between the X-ray detection schemes. This makes it possible to extract a changed portion of a target region such as a lesion and makes it difficult to extract a portion without change as a difference, thereby allowing a doctor or technician to more easily make diagnosis.

As described in processing example 1, it can be assumed that only data that can be obtained by the integral CT apparatus in a past examination is integral tomographic image, no integral projection data or integral volume data can be obtained, and hence registration cannot be performed by using volume data. Processing in such a case will be described with reference to the flowchart of FIG. 3D. Steps S211 to S215 are processing similar to steps S111 to S115 in processing example 1 (FIG. 3B). In step S216, the image processing unit 444 generates a difference image from the pseudo tomographic image and the integral tomographic image registered with each other in step S215. In step S217, the display control unit 446 performs display using the difference image as in step S208.

### Processing Example 3

Processing example 1 described with reference to FIG. 3B and processing example 2 described with reference to FIG. 3D each have exemplified the case where registration is performed by using a pseudo tomographic image unless registration can be performed between volume data. In such processing, in processing examples 1 and 2, a pseudo tomographic image used for registration or the generation of a difference image is obtained from pseudo volume data. In contrast to this, in processing example 3, a pseudo tomographic image used for the registration between a PC type tomographic image and an integral tomographic image and the generation of a difference image is generated from the integral tomographic image. More specifically, in processing example 3, a tomographic image is generated as a pseudo tomographic image which has image quality matching (similar to) the tomographic image obtained by the photon counting scheme and is obtained by improving the image quality of the integral tomographic image obtained in a past examination by image processing. The image processing unit 444 registers an integral tomographic image and a PC type tomographic image and generates a difference image by using the pseudo tomographic image generated from the integral tomographic image in this manner.

FIG. 3E is a flowchart for explaining image processing according to processing example 3. Steps S301 and S302 are similar to steps S111 and S112 (S211 and S212). In step S301, the image processing unit 444 obtains an integral tomographic image that is the first data. Note that integral tomographic images may be stored in a memory (the memory 41 or another memory) in the console apparatus 40 or an external device such as a server connected to the console apparatus 40. In step S302, the image processing unit 444 obtains a PC type tomographic image that is the second data corresponding to the section position of the integral tomographic image obtained in step S301 based on the information of the section position of the integral tomographic image.

In step S303, the image processing unit 444 generates a pseudo tomographic image that is the third data by improving the image quality (for example, graininess or sharpness) of the integral tomographic image obtained in step S301 by image processing. The pseudo tomographic image that is the third data has image quality made similar to the image quality of the tomographic image obtained by the photon counting X-ray detection scheme. For example, the image processing unit 444 obtains a pseudo tomographic image by improving the graininess of the integral tomographic image obtained in step S301 by noise reduction processing and improving the sharpness of the image by image reconstruction processing. For example, linear filter processing using a low pass filter such as a Gaussian filter may be used as noise reduction processing. Alternatively, nonlinear filter processing with image structural conservativeness using an epsilon filter, a bilateral filter, or the like may be used as noise reduction processing. Structural conservativeness type noise reduction processing such as NLmeans or BM3D may be used as another noise reduction processing. In addition, deconvolution processing can be used as image reconstruction processing. In performing deconvolution processing, the modulation transfer functions (MTFs) of a PC type tomographic image (a tomographic image based on the photon counting X-ray detection scheme) and an integral tomographic image (a tomographic image based on the integral X-ray detection scheme) are measured in advance. A tomographic image is generated by applying inverse filter processing to an integral tomographic image so as to match the MTF of a tomographic image based on the PC type X-ray detection scheme. As another image reconstruction processing, a method using a Wiener filter is available. Using Wiener filter processing makes it possible to perform image reconstruction processing while suppressing a deterioration in the graininess of the image. With this processing, a tomographic image (to be referred to as a pseudo tomographic image hereinafter) made similar to the image quality of a PC type tomographic image is obtained. Note that in improving the image quality of an integral tomographic image (image processing for making similar to the image quality of a PC type tomographic image), both or one of the graininess and the sharpness may be improved.

In step S304, the image processing unit 444 registers the PC type tomographic image (the data based on the second data) with the integral tomographic image (the data based on the first data) by using the pseudo tomographic image (the data based on the third data) generated in step S303. The registration processing is similar to step S115. For example, the registration between the pseudo tomographic image and the integral tomographic image is performed by using a method of extracting feature points as in the registration of the volume data described in processing example 1 or a method using a value indicating a correlation. The image processing unit 444 then registers the PC type tomographic image and the integral tomographic image based on the registration result. In step S305, the image processing unit 444 obtains a difference image between the pseudo tomographic image and the PC type tomographic image registered with each other. The difference image represents the difference between the integral tomographic image obtained in a past examination and the PC type tomographic image obtained in the current examination. In step S306, the display control unit 446 displays the integral tomographic image, the PC type tomographic image, and the difference image after the registration. The display processing is similar to the display processing described with reference to steps S208 and S217. Replacing the integral tomographic image with the pseudo tomographic image and displaying it together with the PC type tomographic image allow the operator to compare the tomographic image based on the past examination with the tomographic image based on the current examination which have similar image quality, thereby improving the diagnosis efficiency.

In processing example 3, a pseudo tomographic image similar in image quality to a PC type tomographic image is generated by performing image processing for an integral tomographic image. However, limitation is not made thereto. Obviously, a pseudo tomographic image similar in image quality to the tomographic image that is the second data obtained by the integral scheme may be generated by performing processing (for example, image blurring processing) of reducing at least one of graininess and sharpness with respect to the PC type tomographic image that is the first data. In this case, in step S305, a difference image between the pseudo tomographic image and the integral tomographic image is generated. In addition, in this case, replacing the PC type tomographic image with the pseudo tomographic image and displaying it together with the integral tomographic image allow the examiner to compare the past and current tomographic images with similar image quality.

### Processing Example 4

Processing example 3 has described the arrangement for obtaining a pseudo tomographic image obtained by making the image quality of an integral tomographic image similar to that of a PC type tomographic image by image processing. Processing example 4 will describe an arrangement for obtaining a pseudo tomographic image by making the image quality of an integral tomographic image similar to that of a tomographic image obtained by the photon counting scheme by AI. More specifically, a pseudo tomographic image with image quality similar to that of a PC type tomographic image is obtained by inputting an integral tomographic image obtained in a past examination to a learned model. A difference image between the pseudo tomographic image and the PC type tomographic image is generated by registering the PC type tomographic image with the integral tomographic image by using the pseudo tomographic image. In this case, the learned model is a learning model obtained by machine learning using, as teaching data, a pair of a pseudo tomographic image made similar to the integral tomographic image and the PC type tomographic image (a tomographic image obtained from the photon counting CT apparatus).

FIG. 3F is a flowchart for explaining image processing by processing example 4. Steps S401 and S402 are similar to steps S301 and S302 in processing example 3 (FIG. 3E). In step S403, the image processing unit 444 obtains a pseudo tomographic image that is the third data by inputting the integral tomographic image that is the first data obtained in step S401 to the learned model stored in the storage unit (memory) (not shown) of the control unit 44. The pseudo tomographic image is obtained by improving the image quality of the integral tomographic image to image quality similar to the image quality of the PC type tomographic image.

Learned model construction processing will be described here with reference to FIG. 3G. FIG. 3G is a flowchart for explaining learned model construction processing in processing example 4. In step S411, the image processing unit 444 obtains PC type projection data and generates PC type volume data. In step S412, the image processing unit 444 generates pseudo volume data (pseudo integral volume data) based on a signal detected when obtaining the PC type projection data obtained in step S411. A procedure of generating pseudo volume data is similar to step S103 in processing example 1 (FIG. 3A). That is, the image processing unit 444 obtains the integral value of the product of a photon count and energy from the DAS 18 (or the preprocessing unit 442), obtains pseudo projection data (pseudo integral projection data) similar to the projection data obtained from the integral CT apparatus, and generates pseudo volume data from the pseudo projection data. In step S414, the image processing unit 444 obtains, as teaching data, a pair of the PC type tomographic image obtained from the PC type volume data obtained in step S411 and the pseudo tomographic image obtained from the pseudo volume data. That is, teaching data is obtained by pairing tomographic images at the same tomographic position which are respectively generated from the PC type volume data and the pseudo volume data. In step S415, the image processing unit 444 generates a learned model by providing (training) the machine learning engine with the teaching data obtained in step S414. In this case, many teaching data can be obtained by obtaining pairs of tomographic images at many section positions from the PC type volume data and the pseudo volume data.

The machine learning engine is preferably of a regression type using a convolutional type neural network. This is because this type can make the image quality of one image similar to that of the other image. In addition, as a network, a known network such as Unet, Residual Net, or Dense Net can be used. Alternatively, generative adversarial networks (GAN) or the like may be used. The machine learning engine may include an error detection unit and an updating unit. The error detection unit obtains the error between teaching data and output data that is output from the output layer of a neural network in accordance with the input data input to the input layer. The error detection unit calculates the error between the output data from the neural network and the teaching data by using a loss function. The updating unit updates the combined weighting coefficient or the like between nodes of the neural network so as to reduce the error obtained by the error detection unit based on the error. The updating unit updates the combined weighting coefficient or the like by using, for example, an error backpropagation method. The error backpropagation method is a technique of adjusting the combined weighting coefficient or the like between nodes of each neural network so as to reduce the above error.

Note that a GPU may be used for the machine learning of a learning model and the estimation of a learned model. Increasing the amount of parallel processing using the GPU enables more efficient arithmetic processing and hence is effective in performing learning a plurality of times by using a learning model (for example, deep learning). More specifically, in executing a learning program including a learning model, a CPU and a GPU cooperatively perform arithmetic processing to perform learning. Note that the learning unit may perform arithmetic processing by using only the CPU or the GPU. In addition, a learned model may perform estimation by using the GPU as in the case of learning. With this machine learning, the machine learning engine generates a learned model that outputs a pseudo tomographic image based on an integral tomographic image. In addition, a learned model can be generated for each imaging condition such as a view count, a tube current, and a slice thickness in the same region.

Referring back to FIG. 3F, in step S404, the image processing unit 444 registers the PC type tomographic image (the data based on the second data) with the integral tomographic image (the data based on the first data) by using the pseudo tomographic image (the data based on the third data). This processing is similar to step S304. In step S405, the image processing unit 444 obtains a difference image between the pseudo tomographic image and the PC type tomographic image after the registration. This processing is similar to step S305. In step S406, the display control unit 446 displays the difference image between the PC type tomographic image and the integral tomographic image. This processing is similar to step S306.

### Processing Example 5

In processing examples 2 to 4, a difference region between an integral tomographic image obtained in a past examination and a PC type tomographic image obtained in the current examination is calculated by calculating the difference between the integral tomographic image in the past examination and the pseudo tomographic image corresponding to the PC type tomographic image in the current examination. In contrast to this, in processing example 5, if a region of interest (ROI) is set in one of an obtained integral tomographic image and an obtained PC type tomographic image, the ROI is reflected at the same position in the other tomographic image. This processing will be described below. Note that a region set as an ROI may be an analysis region (a region where an analysis is to be performed). FIG. 3H is a flowchart for explaining the processing performed by the image processing unit 444 and the display control unit 446 in processing example 5.

Steps S501 to S506 are similar to steps S101 to S106 in processing example 1 (FIG. 3A). With the processing so far, the PC type volume data (the data based on the first data) is registered with the integral volume data (the data based on the second data) based on the pseudo volume data (the data based on the third data). The PC type tomographic image and the integral tomographic image registered with each other at the same section position are displayed. The operator can set a region of interest (to be referred to as the first ROI hereinafter) on the PC type tomographic image displayed on the display unit 42 to measure the size and the like of a lesion such as a tumor. In step S507, the input interface 43 as an instruction acceptance unit accepts the setting of the first ROI by an instruction from the operator, the display control unit 446 superimposes information indicating the first ROI (information indicating the first region) on the PC type tomographic image displayed on the display unit 42. Note that in this case, the information indicating the first ROI is a frame on the PC type tomographic image.

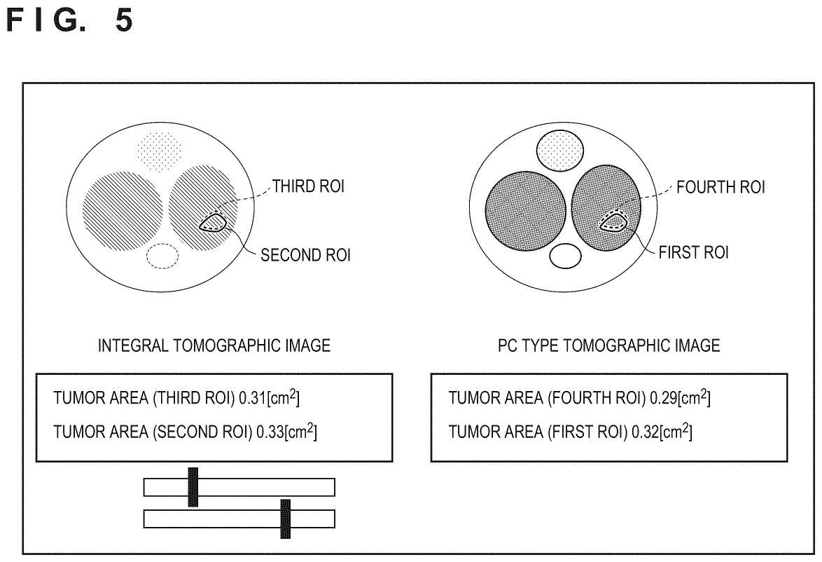

In step S508, the image processing unit 444 calculates a position on the integral tomographic image which corresponds to the first ROI on the PC type tomographic image set by the operator and calculates a position on the integral tomographic image which corresponds to the above position (in other words, sets a corresponding region). The display control unit 446 superimposes the integral tomographic image on information indicating a second ROI (information indicating a second region corresponding to the first region, a frame in this case). Since the integral volume data and the PC type volume data have been registered in step S503, a position for setting the second ROI can be easily calculated. This makes it possible to measure a temporal change by analyzing the same position in follow-up or the like. FIG. 5 shows a display example of information indicating the first ROI and information indicating the second ROI. In this case, for example, if an ROI (third ROI) has already been set in the integral tomographic image, the information indicating the third ROI and the information indicating the second ROI may be displayed in different display forms, for example, displayed in different colors. Displaying the information in different display forms in this manner allows the operator to easily identify the ROI set in the past and the currently set ROI. In addition, the graph or numerical values of measurement values measured in each ROI or analytical values can be displayed in an identifiable manner corresponding to the display form of each ROI. Information indicating a manually set ROI and information indicating an ROI set by the control unit 44 based on an ROI on a tomographic image as a comparison target may be displayed in an identifiable manner by being displayed in different display forms. More specifically, for example, the following pieces of information may be displayed in different colors: information concerning the ROI set by being manually drawn on an integral tomographic image by the operator; and information concerning the second ROI automatically set in the integral tomographic image based on the first ROI set on a PC type tomographic image.

In processing example 5, the second ROI is set on an integral tomographic image based on position information concerning the first ROI set on a PC type tomographic image in the case described above. However, the first ROI may be set on the integral tomographic image, and the second ROI may be set on the PC type tomographic image based on the position information. In this case, the first ROI set on the integral tomographic image may be the third ROI that has already been set in the past. In such a case, as shown in FIG. 5, a region on the PC type tomographic image which corresponds to the third ROI that has already been set on the integral tomographic image in a past examination is specified and is superimposed as information indicating the fourth ROI. If the first ROI is newly set on the PC type tomographic image by the operator, information indicating the third ROI corresponding to the integral tomographic image and information indicating the newly set first ROI may be displayed in an identifiable manner (for example, displayed in different display forms).

In addition, the first to fourth ROIs may be reflected between the tomographic images after the registration in processing examples 3 and 4. For example, in the integral tomographic image and the PC type tomographic image registered with each other by the method of processing example 3, the first ROI set in the PC type tomographic image can be reflected as the second ROI in the integral tomographic image. Likewise, in the integral tomographic image and the PC type tomographic image registered with each other, the third ROI set on the integral tomographic image can be set as the fourth ROI on the PC type tomographic image based on the registration result.

In processing examples 1 to 5, in assumption of a follow-up, the data obtained from the integral CT apparatus and the data obtained from the PC type CT apparatus are those obtained on different dates and times. However, it is not essential that the data should be those obtained on different dates and times. For example, the data may be those obtained by the integral CT apparatus and the PC type CT apparatus on the same date. In addition, in the above processing examples, the projection data and the volume data may be part of the data obtained by the CT apparatuses. The volume data may be partial volume data corresponding to a predetermined organ such as the heart. Processing examples 1 to 5 have been described with reference to the case where the data obtained in a past examination and obtained from the integral CT apparatus is compared with the data obtained in the current examination and obtained from the PC type CT apparatus. However, the present disclosure is not limited to this. For example, even if the integral CT images obtained in the past examination are dual energy CT images obtained by imaging with two or more types of tube voltages can be compared by a similar method. As described above, photon energy information can be obtained from the photon counting X-ray detector 12. Accordingly, it is possible to generate pseudo dual energy CT images similar to the functional images obtained by performing CT imaging with two types of X-rays with different tube voltages and compare them with each other as in processing examples 1 to 5 described above. Note that examples of the functional images include effective atomic number images. In addition, the medical image processing apparatus according to the present disclosure may be capable of performing one or a plurality of types of the types of processing in processing examples 1 to 5. For example, the operator may be allowed to select the processing in steps S111 to S114 in processing example 1 and the processing in steps S401 to S403 in processing example 4. More specifically, the operator can obtain a PC type tomographic image (first data) and an integral tomographic image (second data) and select either the first method of obtaining pseudo volume data similar to the integral volume data from the signal detected by the PC scheme and obtaining a pseudo tomographic image (third data) corresponding to the position of the PC type tomographic image or the second method of obtaining a pseudo tomographic image (fourth data) made similar to the PCCT image by image processing from the integral tomographic image (second data). In this case, the operation to be performed by the operator may be, for example, operating a switching button displayed on a screen displayed by one method or selecting the first method or the second method by a button or pull-down displayed on a screen before the display of an examination result. In the subsequent process of registering the data based on the first data with the data based on the second data by using the third data, steps S115 and S116 or steps S404 to S406 are automatically selected based on a selected method.

As described above, according to processing examples 1 to 5 described above, it is possible to reduce the quality difference between the tomographic image based on the projection data obtained by the photon counting X-ray detection scheme and the tomographic image based on the projection data obtained by the integral X-ray detection scheme. This makes it possible to facilitate accurate registration of these tomographic images and comparison such as observation of a temporal change in the same subject and hence to reduce the load on the doctor or technician and a bottleneck in replacing the apparatus.

According to the disclosure of the present specification, it is possible to further facilitate comparison between the images obtained by different X-ray detection schemes.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions

1. A medical image processing apparatus comprising:
an obtaining unit configured to obtain first data obtained by using a first X-ray detection scheme and second data obtained by using a second X-ray detection scheme different from the first X-ray detection scheme;
a data generation unit configured to generate third data made similar to the second data by processing the first data or a signal for obtaining the first data; and
a registration unit configured to register data based on the first data with data based on the second data based on a result of registration between the data based on the second data and the data based on the third data.

2. The medical image processing apparatus according to claim 1, wherein the processing of the first data or the signal for obtaining the first data is processing for reducing an image quality difference between an image based on the first data and an image based on the second data.

3. The medical image processing apparatus according to claim 1, wherein the first data is data based on count data of X-ray photons collected by using a signal detected by an X-ray detector, and the second data is data based on integral data obtained by integrating a signal detected by the X-ray detector with a predetermined time width.

4. The medical image processing apparatus according to claim 1, wherein the first data is data based on integral data obtained by integrating a signal detected by the X-ray detector with a predetermined time width, and the second data is data based on count data of X-ray photons collected by using a signal detected by the X-ray detector.

5. The medical image processing apparatus according to claim 3, wherein the data generation unit generates the third data based on an integral value obtained by integrating a product of a count of photons and energy included in the count data detected by the X-ray detector.

6. The medical image processing apparatus according to claim 1, wherein the first data and the second data each are projection data or volume data, or one of the first data and the second data is projection data and the other is volume data.

7. The medical image processing apparatus according to claim 1, wherein at least one of the first data and the second data is a tomographic image.

8. The medical image processing apparatus according to claim 3, wherein the data generation unit generates the third data by applying image processing to the first data to reduce graininess or sharpness.

9. The medical image processing apparatus according to claim 3, wherein the data generation unit generates the third data by applying image processing to the first data to improve graininess or sharpness.

10. The medical image processing apparatus according to claim 3, wherein the data generation unit generates the third data from the first data by using a learned model obtained by learning using, as teaching data, a pair of a first image obtained from the first data and a third image obtained from third data.

11. The medical image processing apparatus according to claim 1, further comprising a display control unit configured to display, on a display unit, a first image obtained from data based on the first data and a second image obtained from data based on the second data after the registration.

12. The medical image processing apparatus according to claim 11, wherein the display control unit displays the first image and the second image in parallel or switches and displays the first image and the second image on the display unit.

13. The medical image processing apparatus according to claim 1, further comprising a unit configured to generate an image indicating a difference between a third image obtained from data based on the third data after the registration and a second image obtained from data based on the second data after the registration.

14. The medical image processing apparatus according to claim 13, wherein the image indicating the difference is a difference image indicating a difference region between the third image and the second image.

15. The medical image processing apparatus according to claim 14, wherein the difference image is an image having information indicating the difference region superimposed on a first image obtained from data based on the first data after the registration or the second image.

16. The medical image processing apparatus according to claim 11, further comprising a region setting unit configured to set a second region on the second image which corresponds to a first region in which the first image is set.

17. The medical image processing apparatus according to claim 1, wherein the first data and the second data are data obtained from different X-ray detectors on different dates and times.

18. The medical image processing apparatus according to claim 1, wherein the data generation unit can generate fourth data made similar to the first data by processing the second data or a signal for obtaining the second data and includes an instruction acceptance unit configured to accept a selection instruction for generating one of data based on the third data and data based on the fourth data.

19. A control method for a medical image processing apparatus, the method comprising:
an obtaining step of obtaining first data obtained by using a first X-ray detection scheme and second data obtained by using a second X-ray detection scheme different from the first X-ray detection scheme;
a data generation step of generating third data made similar to the second data by processing the first data or a signal for obtaining the first data; and
a registration step of registering data based on the first data with data based on the second data based on a result of registration between data based on the second data and data based on the third data.

20. A storage medium storing a program for causing a computer to execute:
an obtaining step of obtaining first data obtained by using a first X-ray detection scheme and second data obtained by using a second X-ray detection scheme different from the first X-ray detection scheme;
a data generation step of generating third data made similar to the second data by processing the first data or a signal for obtaining the first data; and
a registration step of registering data based on the first data with data based on the second data based on a result of registration between data based on the second data and data based on the third data.
